SEQUENCE LISTING (1 sequence)

SEQ ID NO: 1 - protein, 764 aa, Homo sapiens
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHEMRQGLHNMEDVYELIENSHLTPKKQGQISEEYMQTVL

(19) United States
(12) Patent Application Publication
(10) Pub. No.: US 2024/0069017 A1
(43) Pub. Date: Feb. 29, 2024

(54) B Cell and Antibody Assays in Autoimmune Diseases and Treatment Thereof

(71) Applicant: Paul Viktor Lehmann, Shaker Heights, OH (US)

(72) Inventor: Paul Viktor Lehmann, Moreland Hills, OH (US)

(73) Assignee: Cellular Technology Limited, Shaker Heights, OH (US)

(21) Appl. No.: 18/493,294

(22) Filed: Oct. 24, 2023

Related U.S. Application Data
(60) Provisional application No. 63/424,670, filed on Nov. 11, 2022.

(51) Int. Cl. G01N 33/564; A61K 31/136; A61K 31/137; A61K 31/785; A61K 38/21; A61P 37/06; C07K 16/28; C12N 5/0781

(52) U.S. Cl. CPC G01N 33/564; A61K 31/136; A61K 31/137; A61K 31/785; A61K 38/215; A61P 37/06; C07K 16/2842; C07K 16/2887; C12N 5/0635; C07K 2317/24; C12N 2503/00

(57) ABSTRACT

Embodiments of this disclosure include methods for detecting autoimmune diseases by detecting the presence of autoantibodies directed against self-antigens (or “respective antoantigens”). In particular, embodiments of this disclosure include detecting autoantibodies produced by B cells from a patient suspected of having an autoimmune disease using a Direct B Cell test. Other embodiments include detecting autoantibodies produced by memory B cells using a Memory B Cell test. Further embodiments include methods of treatment of a patient having an autoimmune disease comprising administering a substance that decreases B cell numbers. Other embodiments comprise treating a patient with other known treatments.

### CLAIM OF PRIORITY

This application claims priority to U.S. provisional application No. 63/424,670 filed 11 Nov. 2022, and entitled: “B Cell and Antibody Assays in Autoimmune Diseases and Treatment Thereof,” Paul Viktor Lehmann, inventor. This application is incorporated herein fully by reference.

### SEQUENCE LISTING

The Sequence Listing in ST.26, version 1.6, approved Nov. 25, 2022, format written in file CLTL-1046US2_ST26.xml, is filed under 37 C.F.R. 1.831-1.839, the sequence listing created 19 Oct. 2023, 2,776 bytes, machine format IBM-PC, MS-Windows operating system, is hereby incorporated by reference in its entirety and for all purposes.

### FIELD OF THE INVENTION

This invention relates generally to assays for antibodies directed toward antigens involved in B Cell mediated autoimmune diseases, and their uses in diagnosis, prognosis, and treatment of such diseases. Particularly, this invention relates to methods and assays for immune cells and antibodies as diagnostic, prognostic or therapeutic biomarkers of autoimmune diseases. More particularly, this invention relates to detection in the blood of B lymphocytes and antibodies that bind to antigens involved in autoimmune diseases.

### BACKGROUND

There are several autoimmune diseases that afflict many human beings. For example, Grave's disease (GD), myasthenia gravis (MG), muscular dystrophy (MD), systemic lupus erythematosus (SLE), rheumatoid arthritis (RA), celiac disease (CD), type-1 diabetes (T-1D), Guillain-Barre syndrome (GBS), Hashimoto's thyroiditis (HT), polymyalgia rheumatica (PR), alopecia areata (AA), antiphospholipid syndrome (APS), eosinophilic esophagitis (EE), and multiple sclerosis (MS) are considered to be autoimmune diseases of different organ systems. These diseases can be classified into larger groups of diseases of: connective tissue, autoimmune vasculitis and APS, antithyroid, gastrointestinal immune-related adverse events, hepatic, autoimmune nephrology, autoimmune dermatology, and autoimmune neuropathies.

### SUMMARY

Assessment of humoral immunity is commonly confined to measurements of serum antibody reactivity. These pre-formed immunoglobulin molecules can convey immunity by preventing a re-infection. In some instances, however, a re-infection does occur, and in such cases, it is the mobilization of an anamnestic B and T cell response that confers immune protection. Relying on fundamentally different mechanisms, therefore, passive immunity conveyed by pre-existing antibodies needs to be distinguished from active B cell memory.

In the course of a primary B cell response, naïve B cells proliferate, undergo immunoglobulin class switching and affinity maturation while they differentiate into antibody-secreting cells (ASC, the effectors of the B cell system), or memory B cells. Plasma cells are prevalent ASC in vivo. Plasma cells can be short- or long-lived and their survival depends on competition for niches in the bone marrow.

Previously, we detected neuroantigen-specific B [symbol] , in the absence of detectable serum antibody levels in most patients, whereas such B [symbol] were absent in healthy controls. Collectively these findings suggest that detecting B [symbol] -derived ASC might be a more reliable way to diagnose past infections and possibly also autoimmune diseases than afforded presently by serum antibody measurements (Lehmann et al., Monitoring memory B cells by next generation ImmunoSpot provides insights into humoral immunity that measurements of circulating antibodies do not reveal, https.//doi.org/10.2203/rs.3.rs-3414085/v2).

Assessment of humoral immunity by measurement of antibody levels does not reflect on memory B cell frequencies, and thus an individual's potential to engage into an anamnestic antibody response against the same or an antigenically-related virus. Assessment of memory B cells can provide insights into humoral immunity that measurements of serum antibodies do not convey. Direct monitoring of the antigen-reactive memory B cell compartment is both required and feasible towards that goal.

Long-lived memory B cells are also generated during the course of a primary B cell response. Similar to their naïve precursor cells, memory B cells are also resting lymphocytes that re-circulate via the blood throughout the lymphoid tissues of the body. Importantly, upon antigen re-encounter memory B cells undergo rapid and robust proliferation and give rise to new generations of daughter cell progeny that include effector cells (ASC) and more memory B cells. However, unlike their naïve precursor cells, memory B cells are present in increased numbers in the body due to their clonally expanded status, and have already undergone immunoglobulin class switching and affinity maturation. Memory B cells can therefore, be found in the peripheral blood, and samples of blood contain peripheral blood mononuclear cells (PBMCs) some of which are memory B cells.

Diagnosis, prognosis and evaluation of therapy of autoimmune diseases can be made using cell-based in vitro assays, including enzyme-linked immunospot (ELISPOT), or enzyme-linked immunosorbent (ELISA) assay methods. To detect anti-self-antibodies, a sample of blood is drawn from a patient suspected of having an autoimmune disease. Then, peripheral blood monocytes (PBMCs) are prepared.

In general, phrased for all antibody-mediated autoimmune diseases (these all share the same pathomechanism), detecting the autoantigen-specific memory B cells is a more reliable for diagnosing and monitoring an antibody-mediated autoimmune disease than detecting the corresponding autoantigen-specific antibodies in serum.

In one specific version, e.g. for myasthenia gravis (MG), detecting acetyl choline receptor (ACHR, the autoantigen targeted in MG)-specific memory B cells is a more reliable for diagnosing and monitoring of MG (an antibody-mediated autoimmune disease) than detecting ACHR-specific antibodies in serum.

Detecting memory B cells can be accomplished, either (a) B cell ELISPOT, or (b) fluorescent antigen probe staining of B cells followed by flow cytometry, or (c) detecting the antibody in the cell culture supernatant of polyclonally stimulated (polyclonally activated) B cells.

To accomplish these objectives, B cell ELISPOT can be done in two ways.
(1) An autoantigen itself is coated onto the membrane directly, or using an affinity coating approach such as genetically encoded hexahistigine (6×his) affinity tags. The polyclonaly stimulated (polyclonally activated) B cells are plated on top of the membrane. B cells that secrete antigen-specific antibody leave an antibody footprint on the membrane that is visualized.
(2) A membrane is coated with a reagent that is specific for human antibodies (e.g., an anti-human IgG). When polyclonally stimulated B cells are now added, every antibody secreting B cell leaves a secretory footprint, irrespective of the B cell's specificity. As the next step, however, tagged antigen is added that binds only where antigen-specific B cells left an antibody footprint. Now the plate-bound antigen is detected via its tag.

Like in a prior multiple sclerosis patent (U.S. Pat. No. 11,340,224), the B cells can be tested direct ex vivo (the direct assay—revealing in vivo activated B cells seen only when the disease is flaring) or after polyclonal stimulation (polyclonally activated, detecting antibodies produced by memory B cells).

### DETAILED DESCRIPTION

### Definitions

The following definitions are included for convenience. The meanings of the following terms are to be defined immediately below, unless such term(s) are defined specifically in the sections that follow.

“ANA” means antinuclear antibodies.

“ENA” means an autoimmune disease characterized by presence of autoantibodies against the antigens dsDNA (double stranded DNA), chromatin, ribosomal-P protein, SS-A, SS-B, Sm, Sm/RNP, RNP, Scl-70, Jo-1, and centromere B.

“AP” means alkaline phosphatase.

“APC” or “APCs” means antigen presenting cells.

“Autoimmune myositis” means an autoimmune disease characterized by presence of autoantibodies against one or more of the antigens dsDNA, chromatin, ribosomal-P protein, SS-A (Sjrogen's disease), SS-B (extractable nuclear antigen; Sjogren's disease and lupus erythermatosus), Sm (ceramide/sphingomyelin), Sm/RNP (ceramide/sphyngomyelin ribonucleoprotein), RNP (ribonucleoprotein), Scl-70 (topoisomerase I; systemic lupus erythrematosus), Jo-1 (histidyl-tRNA synthetase), and centromere B (found in patients with scleroderma and Ranaud's dissease).

“RF, CCP” means immunoassays characterized by presence of IgG, IgM and IgA antibodies.

“Systemic sclerosis” means an autoimmune disease characterized by presence of autoantibodies against one or more of the antigens Scl-70, CENP A, CENP B, RP11, RP155, Fibrillarin, NOR90, Th/To, PM-Scl100, PM-Sc175, Ku, PDGFR, or Ro-52.ANCA

“ANCA” means an autoimmune disease characterized by presence of autoantibodies against one or more antigens the cytoplasmic neutrophils pANCA, cANCA, X-ANCA, PR3, MPO or GBM.

“AECA” means anti-endothelial cell antibodies.

“ASCA” means anti-Saccharomyces cerevisiae antibodies.

“Anti-cardiolipin antibodies” means cCL, IgG, or aCL, or IgM antibodies.

“Lupus anticoagulant” means phospholipid antibodies against anti-beta-2 glycoprotein 1.

“TPO” means thyroid peroxidase antibodies.

“TPO, TRAb or TgAb” means autoantibodies against thyroid tissue.

“tTG” or “gliadin” antibody means autoantibodies characterized in celiac disease.

“IBD” means inflammatory bowel disease, characterized by autoantibodies directed against antigens ANCA, ASCA, OMP, DGP, A4-Fla2 or CBirl.

“OMP and OMPA” means heat-modifiable, cell surface porin proteins.

“DGP” means deamidated gliadin, a protein found in gluten.

“A4-Fla2” means a dominant flagellin antigen found in Crohn's disease.

“Fla-X” means a flagellin antigen found in Crohn's disease.

“CBirl” means a flagellin antigen found in Crohn's disease.

“Actin” means a protein found in muscles and the liver and is autoantibodies against actin are found in hepatic autoimmunity.

“AMA M2” means an antigen in mitochondria; autoantibodies against AMA M2 are found in patients with autoimmune hepatitis (AIH).

“M2-3E” means a recombinant fusion protein consisting of different subunits of the mitochondrial enzyme complex M2; antibodies against M2-3E are common in patients having primary biliary cholangitis (PBC).

“LKM-1” means liver kidney microsome protein type 1; autoantibodies against LKM-1 recognize cytochrome P450 2D6 (CYP2D6) and is associated with type 2 autoimmune hepatitis.

“LC-1” means a liver cytosolic protein 1 and is within the multiform liver/kidney microsomal LKM family and is found in type 2 autoimmune hepatitis.

“SLA/LP” means a soluble liver antigen/liver-pancreas; autoantibodies against UGA repressor t-RNA associated protein are found in patients with autoimmune hepatitis.

“SP100” means speckled protein 100 a subnuclear organelle and component of the promyelocytic leukemia nuclear bodies and is found in patients with hepatic autoimmune hepatitis.

“PML” means progressive multifocal leukoencephalopathy; autoantibodies against myelin can cause multiple sclerosis-like lesions.

“gp210” means a nuclear protein; autoantibodies against gp210 is associated with primary biliary cholangitis (PBC).

“PLA2R type M and N” mean phospholipase A2 receptor; autoantibodies against PLA2R are associated with persistence of nephrotic proteinuria.

“Envoplakin” and “periplakin” mean members of the plakin family of cytoskeletal linker proteins; autoantibodies against these plakins are associated with autoimmune dermatitis.

“Desmoglein 1” and “Desmoglein 3” mean members of the desmoglein protein subfamily, are located on chromosome 18q2, and are cadherin-like transmembrane glycoproteins in the desmosome (cell-cell junctions); autoantibodies against desmogleins result in loss of structural strength and are found in patients with autoimmune dermatitis

“BP230-CF” means an extracellular membrane protein, and “BP180-NC16A-4” means BP180 has a linker NC16A-4 anchoring the proteins to the cellular membranes in the esophagus and skin; autoantibodies against BP230-CF or BP180-NC16A-4 are found in patients with bullous pemphigoid.

“GM1,” means a monosialotetrahexysolganglioside; autoantibodies against GM1 are found in patients with autoimmune neuropathies.

“GM2,” means a urinary GM2-activator protein (GM2AP); autoantibodies against GM2 are markers for lung cancer.

“GM3,” means a gangloioside in serum and is associated with serum lipoproteins; it is involved in the barrier function of endothelial cells.

“GCla,” “GD1b,” “GT1b,” and “GQ1b” are ganglioside proteins.

“Amphiphysin/DV2.1” means a brain-enriched protein; autoantibodies can be found in patients with breast cancer or stiff person syndrome.

“Recoverin” means photoreceptor cells Ca (2+)-binding protein.

“Hu” means proteins clustered in the nuclei of neurons; anti-Hu (Anti-ANNA1) antibodies can be found in patients with Anti-ANNA1 associated encephalitis

“SOX1,” “SOXs,” and “SOX3” mean genes that encodes a transcription factor with HMG-box (high mobility group) DNA-binding domain; anti-SOX antibodies can be found in patients with Epilepsy.

“Titin” means a protein encoded by the TTN gene; acts like a molecular spring to determine the length of muscle fiber; found in patients with myositis.

“Yo” means “Yo-PCD;” a neural antigen; antibodies can be found in patients with ovarian cancer and paraneoplastic cerebellar degeneration.

“CV2” means SARS-CoV-2; found in patients with COVID-19.

“Mall/2” means proteins that reduce antigen presentation on tumor cells.

“PNMA2” means paraneoplastic antigen Ma2; autoantibodies can be found in patients with neuroendocrine tumors.

“PCA-2” means Purkinje cell cytoplasmic antibody type 2; PCA-2 binds to a cytoplasmic antigen in neurons and SCLC (small cell lung carcinoma) cells; found in patients with limbic encephalitis, cerebellar ataxia myasthenic syndrome.

“AGNA” means anti-glial nuclear antibody.

“CARPVIII” means carbonic anhydrase-related protein; found in Purkinje cells; found in patients with paraneoplastic cerebellar degeneration.

“Tau” means a protein known as tubulin associated unit maintains microtubules in axons; anti Tau antibodies can be found in patients with neurofibrillary degeneration).

“Beta-amyloid” means a protein that accumulates in grains of patients with Alzheimer's disease.

“ApoE4” means a protein Apolipoprotein E; found in patients with Alzheimer's disease.

“BASE1” means

“Neurogranin” means a protein expressed in granule-like structures in pyramidal cells in the hippocampus and cortex; marker of cognitive decline and biomarker of Alzheimer's disease.

“pNf-H” means a protein phosphorylated neurofilament heavy subunit; a biomarker for amyotrophic lateral sclerosis)

“alpha-synuclein” means a protein found in Lewy bodies of patients with Parkinson's disease.

“BBB” means blood brain barrier.

“B-cells” means B-lymphocytes, including plasmablasts, plasma cells, effector B-cells and memory B-cells.

“β-mercaptoethanol”, “2-mercaptoethanol”, “2-ME” and “β-ME” means the chemical compound with the formula HOCH₂CH₂SH. It can be used for the polyclonal stimulation of B lymphocytes.

“Blood antibodies” means antibodies present in blood/serum, including antibodies against self-antigens produced by white blood cells, in particular B lymphocytes.

“CNS antigen” means proteins of the central nervous system (CNS), in particular antigens including myelin antigens such as proteolipid protein (PLP), myelin basic protein (MBP) or myelin oligodendrocyte glycoprotein (MOG), but also neuronal antigens and additionally, fusion proteins, recombinant proteins or analogues of these proteins, the analogues being defined by their cross-reactivity with the respective antigen-specific antibodies. CNS antigens also include peptides that contain sequences of the protein CNS antigens.

“CNS lysate” means a solution produced when the CNS tissue is disrupted in a process known as lysis. This releases the contents of the tissue. After a crude lysate has been generated, the different components can be separated such as organelles, membrane lipids, proteins and nucleic acids. In this application “CNS lysate” includes proteins derived from CNS tissue. CNS lysate is commercially available or can be prepared following the standard procedures. An example of such a protocol would be to remove the CNS tissue, freeze it in liquid nitrogen and keep it at −70° C. or below until further use. Ice-cold lysis buffer containing Tris, EDTA, Triton X-100 and protease inhibitors is added to the frozen tissue, which is then homogenized on ice.

Subsequently, the homogenate is ultracentrifuged and the supernatant isolated. The protein concentration can be determined e.g., by Bradford assay. The supernatant is then stored at −70° C. or below until further use.

“CD” means cluster of differentiation and refers to cell surface molecules on a cell classifying this cell.

“CFA” means complete Freund's adjuvant and is a mixture of IFA and M. tuberculosis strain H37RA.

“CIS” means clinically-isolated syndrome.

“DNA” means deoxyribonucleic acid.

“drLNs” means draining lymph nodes.

“ELISA” means enzyme-linked immunosorbent assay.

“ELISPOT” means enzyme-linked immunosorbent spot technique.

“FBS” means fetal bovine serum.

“FITC” means fluorescein isothiocyanate.

“HL-1” means a chemically defined culture medium containing less than 30 μg protein per ml.

“HRP” means horseradish peroxidase.

“IFA” means incomplete Freund's adjuvant and is a mixture of mannide monooleate and paraffin oil.

“Ig” and “IgG” mean immunoglobulin=antibody.

“IL” means interleukin.

“MHC” means major histocompatability antigens

“MOG” means nyelin oligodendrocyte glycoprotein.

“MOGp” means MOG peptide 35-55.

“MP4” means MBP-PLP fusion protein.

“MRI” means magnetic resonance imaging.

“MS” means multiple sclerosis.

“pVMT” means a B cell-deficient strain of laboratory mice.

“NMO” means neuromyelitis optica.

“PBMC” means peripheral blood mnononuclear cells.

“PBL” means peripheral blood lymphocytes.

“PLP” means proteolipid protein.

“PLPp” means PLP peptide 139-151.

“PPMS” means primary progressive multiple sclerosis. PPMS is characterized by a gradual progression of the disease from its onset with no superimposed relapses and remissions.

There may be periods of a leveling off of disease activity. PPMS differs from RRMS and SPMS in that the onset is typically in the late thirties or early forties, men are as likely women to develop it and initial disease activity is often in the spinal cord and not in the brain. Primary Progressive MS often migrates into the brain, but is less likely to damage brain areas compared to RRMS and SPMS—for example, people with PPMS are less likely to develop cognitive deficits. PPMS is the subtype of MS that is least likely to show inflammatory (gadolinium enhancing) lesions on MRI scans. The primary progressive form of the disease affects between 10 and 15% of all people with multiple sclerosis.

“PTx” means pertussis toxin.

“Polyclonal Stimulation” or “Polyclonal Activation” mean exposing a sample of cells or blood to a substance that stimulates the proliferation of immune cells such as memory B cells and transitioning them to ASC by use of a polyclonal stimulator or polyclonal activator. Examples of such substances are R848, Interleukin-2 (IL-2) and f1-mercaptoethanol also known as 2-mercaptoethanol, and Human-Poly-S containing resiquimod and recombinant human IL-2.

“R848” and “Resiquimod” means an imidazoquinoline compound which activates immune cells as an agonist acting via the TLR7/TLR8 MyD88-dependent signaling pathway. R848 was shown to trigger NF-κB activation in cells expressing murine TLR8 when combined with poly(dT). It is used for the polyclonal stimulation of B lymphocytes.

“RPMI-1640” means a cell culture medium for leukocytes and other cell types, RPMI stands for Roswell Park Memorial Institute, where RPMI was developed.

“RIS” means radiologically-isolated syndrome.

“T-cells” means all T effector cell lineages including Th1 and/or Th2- and/or Th9- and/or Th17- and/or Th22-lymphocytes.

“TCR” means T cell receptor.

“T_{H}” means T helper and refers to different types of T-cells.

“TMB” means 3,3′,5,5′-Tetramethylbenzidine.

“WT” means wild type.

“TSHR” means thyroid stimulating hormone receptor.

“ACC” means “affinity capture coating.” ACC can be used by pre-conditioning an assay membrane with an antibody specific for a genetically-encoded hexahistidine (6×His) affinity tag. This enables high-affinity capture of any 6×His-tagged protein, resulting in detection of rare antigen-reactive memory B cells.

“6×His” means an affinity tag of a recombinant protein that enables subsequent high-affinity capture of any 6×His-tagged protein.

### Autoimmune Diseases

There are numerous autoimmune diseases. These diseases are the result of autoantibodies produced in a person suffering from an autoimmune disease. Generally, the body differentiates between “self” antigens, and “foreign” antigens. Thus, in normal subjects, antibodies are produced against foreign antigens, but not against self-antigens. In autoimmune diseases, the immune system may recognize a self-antigen (or “respective autoantigen”) as foreign and mounts an immune response against the self-antigen. Such immune responses may result in damage to the cell, tissue, or organ having the self-antigen and may result in destruction of the normal function of the cell, tissue, or organ, and in serious cases, can result in morbidity or mortality. Autoimmune diseases may afflict many cells, tissues, and organs.

In general, for all antibody-mediated autoimmune diseases (which all share the same pathomechanism), detecting the autoantigen-specific memory B cells is a more reliable for diagnosing and monitoring an antibody-mediated autoimmune disease than detecting the corresponding autoantigen-specific antibodies in serum.

A specific version, e.g. for myasthenia gravis (MG), detecting acetyl choline receptor (ACHR, the autoantigen targeted in MG)-specific memory B cells is a more reliable for diagnosing and monitoring of MG (an antibody-mediated autoimmune disease) than detecting ACHR-specific antibodies in serum.

Detecting memory B cells can be accomplished, either (a) B cell ELISPOT, or (b) fluorescent antigen probe staining of B cells followed by flow cytometry, or (c) detecting the antibody in the cell culture supernatant of polyclonally stimulated (polyclonally activated) B cells.

To accomplish these objectives, B cell ELISPOT can be done in two ways.
(1) An autoantigen itself is coated onto the membrane directly, or using an affinity coating approach and the polyclonaly stimulated B cells are plated on top. B cells that secrete antigen-specific antibody leave an antibody footprint on the membrane that is visualized.
(2) A membrane is coated with a reagent that is specific for human antibodies (e.g., an anti-human IgG). When polyclonally stimulated B cells are now added, every antibody secreting B cell leaves a secretory footprint, irrespective of the B cell's specificity. As the next step, however, tagged antigen is added that binds only where antigen-specific B cells left an antibody footprint. Now the plate-bound antigen is detected via its tag.

Like in our multiple sclerosis patent U.S. Pat. No. 11,340,224, the B cells can be tested direct ex vivo (the direct assay—revealing in vivo activated B cells seen only when the disease is flaring) or after polyclonal stimulation using an indirect assay.

In some embodiments, PBMCs can be harvested from tissue culture flasks and washed with PBS prior to counting using Live/Dead cell counting suite on an ImmunoSpot® Ultimate S6 Analyzer (Cellular Technology Limited, Shaker Heights Ohio). Cell pellets can be resuspended at 1-3×10⁶ live cells per mL (when measuring antigen-reactive IgG⁺ASC) or 3×10⁵ live cells per mL (for detection of all IgG-secreting cells) in complete BCM and seeded into ImmunoSpot® assays.

For enumeration of all IgG-secreting cells (total IgG⁺ASC), the cell suspensions can be serially diluted 2-fold in duplicates, starting at 3×10⁴ live cells/well, in round-bottom 96-well tissue culture plates (Corning, Sigma-Aldrich). Subsequently, cells were transferred into ImmunoSpot® assay plates that were pre-coated with anti-κ/λ capture antibody reagents (from CTL) and incubated for 16 h at 37° C., 5% CO2. Plate-bound immunoglobulin (Ig) spot-forming units (SFU) can be subsequently visualized using a human IgG-detecting ImmunoSpot® kit (from CTL) according to the manufacturer's instructions. For enumeration of antigen-reactive IgG⁺ASC, ImmunoSpot® assays were performed with ACC as previously described in Koppert, S., et al., Affinity Tag Coating Enables Reliable Detection of Antigen-Specific B Cells in ImmunoSpot Assays. Cells, 10(8) 2021. Briefly, assay plates were first pre-conditioned with 70% (v/v) EtOH followed by two washing steps with PBS. Next, wells were coated with purified anti-6×His tag antibody (BioLegend) at 10 μg/mL in PBS overnight at 4° C. The following day, assay plates were washed once with PBS and then coated overnight with 6×His-tag labeled recombinant protein at 10 μg/mL in PBS. After overnight incubation of the 6×His-tagged recombinant protein coating solutions at 4° C., plates were washed once with PBS and then blocked with complete BCM for 1 h at room temperature prior to addition of polyclonally-stimulated PBMC at the specified cell numbers per well. Plates can then be incubated at 37° C., 5% CO₂ or 16 h and SFU can be subsequently visualized using the human IgG-detecting ImmunoSpot® kit (from CTL) according to the manufacturer's instructions.

### Categories of Autoimmune Diseases

In general, autoimmune diseases and some autoimmune markers can be categorized as follows, with the autoimmune markers that can be used to identify them.

I-A. Connective Tissue Disease Evaluation
I-A-1. ANA screen by IFA with reflex to ENA for all positive samples
I-A-2. ENA identification by immunoassay (dsDNA, chromatin, ribosomal-P protein, SS-A, SS-B, Sm, Sm/RNP, RNP, Scl-70, Jo-1, and centromere B)
I-A-3. RF, CCP by immunoassay (IgG, IgM and IgA)
I-A-4. Autoimmune myositis profile (Mi-2, Ku, PM-Scl100, PM-Sc175, Jo-1, SRP, PL-7, PL-12, EJ, OJ, Ro-52)
I-A-5. Systemic Sclerosis Profile (Scl-70, CENP A, CENP B, RP11, RP155, Fibrillarin, NOR90, Th/To, PM-Scl100, PM-Sc175, Ku, PDGFR, Ro-52)

I-B. Autoimmune Vasculitis and APS
I-B-1. ANCA screen by IFA with reflex to immunoassay for all positive samples (pANCA, cANCA, X-ANCA)
I-B-2. PR3, MPO and GBM antibody identification by immunoassay
I-B-3. Anti-endothelial cell antibodies (AECA)
I-B-4. Anti-cardiolipin antibodies (aCL IgG and aCL IgM)
I-B-5. Phospholipid antibodies (lupus anticoagulant, anti-beta-2 glycoprotein 1)

I-C. Antithyroid Autoantibodies
I-C-1. TPOAb, TRAb and TgAb

I-D. Gastrointestinal Immune-Related Ddverse Events
I-D-1. Celiac disease screen (tTG and gliadin antibody)
I-D-2. IBD screening (ANCA, ASCA, OMP, DGP, A4-Fla2, CBir1)

I-E. Hepatic Autoimmunity (hepatitic and cholangitic patterns of injury)
I-E-1. Liver autoantibodies (Actin, AMA M2, M2-3E, LKM-1, LC-1, SLA/LP, Sp100, PML, gp210, SLA/LP)

I-F. Autoimmune Nephrology Profile
I-F-1. PLA2R type M and N

I-G. Autoimmune Dermatology Profile
I-G-1. Envoplakin, Desmoglein 1, Desmoglein 3, BP230-CF, BP180-NC16A-4
I-G-2. Prickle cell desmosomes, Epidermal basement membrane, Bullous pemphigoid antigens, Keratin (filaggrin, “RA keratin”), Gliadin (GAF-3X), Collagen type VII NC1

I-H. Autoimmune Neuropathies
I-H-1. Ganglioside profile (GM1, GM2, GM3, GD1a, GD1b, GT1b, GQ1b)
I-H-2. Neuronal antigens profile (Amphiphysin/CV2.1, Recoverin, Hu, Yo, Ri, SOX1, titin)
I-H-3. Multiple sclerosis screen (oligoclonal IgG bands, IgG index, Albumin ratio; isoelectrofocusing)
I-H-4. Paraneoplastic syndrome screen (Yo, Hu, Ri, CV2, Ma1/2, amphiphysin, PNMA2, Zic4, GAD65, Tr. Recoverin, SOX1, PCA-2, AGNA, CARPVIII; ELISA, Western blot, IFA)
I-H-5. Neurodegenerative diseases (Tau, Beta-amyloid, ApoE4, BASE1, neurogranin, pNf-H, Alpha-synuclein; ELISA)
I-H-5. Demyelination (NMOSD and MS) (AQP-4, MOG, Flotillin, MAG, gangliosides)

### Detection of Antibodies Against Autoimmune Antigens

A useful tool for detecting antibodies related to autoimmune diseases includes enzyme-linked immunospot (ELISPOT) assays, and enzyme-linked immunosorbent (ELISA) assays.

PBMCs are plated at 2×10⁵ cells per well into 96 well microtiter plates containing the hydrophobic membrane precoated with an antibody as capture reagent. The cells are incubated in the wells with antigen for 24 h at 37° C., 8% CO₂. Peripheral blood mononuclear cells (PBMCs) are isolated from the blood, e.g., by density gradient centrifugation. The memory B cells contained in the sample are polyclonally stimulated e.g., with IL-2, R-848 and 0-mercaptoethanol and subsequently transferred to filter plates or ELISA plates that have been coated with self-antigen. The polyclonally stimulated memory B Cells are then incubated on the plate. After suitable incubation time, allowing sufficient binding of secreted antibodies to the antigen on the plate, the production of antibodies to autoantigens are detected by either fluorescent or colorimetric enzyme detection. Antibodies measured include IgM, IgD, IgG and IgG isotypes.

In general, detection of antibodies related to autoimmune diseases can be accomplished by attaching an autoantigen related to the disease onto the surface of a plate, and then adding a sample of peripheral blood mononuclear cells (PBMCs) that contain memory B cells to the plate. After a period of time, the B cells secrete antibodies and if such antibodies bind to the antigen, the antibodies can be visualized using a secondary antibody (e.g., anti-IgG antibody) linked to an enzyme, colored substrate, or a radioactive marker, rendering the bound antibody visible under a microscope (ELISPOT) assay.

Alternatively, in solution an antibody can be detected by use of ELISA methods.

There are generally several types of B cell assays useful. They include the “Direct B Cell test, the “Indirect B Cell Test,” bead array tests, and ELISA tests described below.

Direct B Cell Test

Plasmablasts, also known as plasma cells or effector B-cells are characterized by the production of antibodies. If plasmablasts, also known as plasma cells, or effector B-cells produce antibodies that are specific for a self-antigen they will produce antibodies against their specific antigen, which can be directly measured in the blood. Measurement of such antibodies can be detected using methods developed by Cellular Technology Ltd. (Neurospot™ is a trademark of Cellular Technology Ltd, Shaker Heights OH).

In cases in which the B cells are actively producing anti-self-antibodies the PBMCs can be plated without any polyclonal activator. The B cells secrete antibodies where they are located on the surface of the assay plate, and when the anti-self-antibodies bind to the antigens on the plate, and subsequently, adding a detector molecule, such as anti IgG coupled to a detector moiety (fluorescent tag, colored bead, or radioactive moiety), and can be detected using a microscope.

In some embodiments a high affinity method (ACC; affinity capture coating) can increase the ability to detect rare B memory cells. ACC can be accomplished by pre-conditioning an assay membrane with an antibody specific for a genetically-encoded hexahistidine (6×His) affinity tag. This enables high-affinity capture of any 6×His-tagged protein, resulting in detection of rare antigen-reactive B cells.

Memory B Cell Test

Memory B cells may require several days of stimulation with polyclonal activators before they start secreting antibodies. In cases in which the B cells in the PBMCs are not actively secreting anti-self-antibodies, one can use a polyclonal activator (polyclonal stimulator), such as interleukin-2 and/or R-848, and/or β-mercaptoethanol to clonally expand the memory B cells (“Indirect B-cell” assay).

Freshly thawed PBMC samples can be resuspended in B cell medium (BCM) containing RPMI 1640 (Lonza, Walkersville, MD) supplemented with 10% fetal bovine serum (Gemini Bioproducts, West Sacramento, CA), 100 U/mL penicillin, 100 U/mL streptomycin, 2 mM L-Glutamine, 1 mM sodium pyruvate, 8 mM HEPES (all from Life Technologies, Grand Island, NY) and 50 μM β-mercaptoethanol (Sigma-Aldrich, St. Louis, MO).

PBMCs can then be stimulated with Human B-Poly-S containing resiquimod and recombinant IL-2 (CTL) at 2×10⁶ cells/mL in 25 cm² tissue culture Such memory B cells are programmed in the body to be able to produce antibodies against antigens. After B-cell stimulation in vitro with polyclonal activators, the B-cells within the PBMC become activated and start producing antibodies. In a subsequent readout system that detects self-antibody producing B-cells, one can identify the number of B-cells and can quantify the magnitude of the responses. In patients with acute disease and recent immune activation, B-cells secreting self-antigen-specific antibodies can be directly detected in the blood without any polyclonal activator (“Direct B-cell” assay). In some experiments described herein, membranes in or the surface of cell culture wells, are coated with self-antigens, either native purified, or recombinant antigens.

When polyclonally activated B-cells are seeded on top of the self-antigen-coated membrane, the antibodies produced by self-antigen-specific B-cells will bind to the membrane around the secreting B-cell and the individual spot forming cells can be enumerated via the ELISPOT technology thus revealing the frequency of self-antigen-specific B-cells in the cell population of activated PBMCs. Antibodies produced by B-cells with other specificity are not bound to the membrane and go undetected. ELISPOT detection is a highly quantitative and sensitive detection system, other detection systems well suited to detect antigen-specific antibodies before or after polyclonal activation of B-cells can also be used. These assays include ELISA, cytokine bead arrays, immune staining, immunoblotting, immune precipitation and immune agglutination.

In some embodiments a high affinity method (ACC; affinity capture coating) can increase the ability to detect rare B memory cells. ACC can be accomplished by pre-conditioning an assay membrane with an antibody specific for a genetically-encoded hexahistidine (6×His) affinity tag. This enables high-affinity capture of any 6×His-tagged protein, resulting in detection of rare antigen-reactive memory B cells.

Bead Array Based Assays

Use of Detection Surfaces in Detection Particle Bead Array Based Assay

Bead array technology is described in U.S. Pat. Nos. 7,598,093 and 8,088,630, both herein incorporated fully by reference. Briefly, a detection surface is in a cell culture plate (e.g., 96-well or 360-well microtiter plate) to detect cellular products. First, the detection surface can be the surface of a well in the plate or a membrane, which is coated with an antibody specific for the analyte of interest as a capture reagent. After several hours of incubation (2 hrs to overnight), during which the capture reagent(s) bind(s) to the membrane, excess capture reagent is washed away. The unsaturated surfaces of the membrane are then blocked with irrelevant protein (bovine serum albumin or gelatin) to prevent subsequent nonspecific binding of analytes. Following the blocking step, the plates are washed to remove non-plate bound, excess blocking reagent. In some embodiments, 6×His tag labeled protein self-antigens can be attached to an assay surface.

At this point the detection substrate is properly prepared to test cells. In some embodiments, human peripheral blood mononuclear lymphocytes (PBMCs) are added at a concentration of for example, about 5×10⁵ per well. Self-antigen is added to the experimental wells, control wells contain no antigen or irrelevant antigen (e.g., myohemerythrin, a protein that humans have not encountered). Among all cells plated (about 5×10⁵ cells) only the self-antigen specific B-cells will be stimulated by the antigen to release secretory products anti-self-antibodies. In the control wells, in the absence of the antigen, antigen specific cells are not stimulated and do not release secretory products. During a 4-48 hour cell culture period (dependent on the product to be detected), the self-antigen specific B cells secrete their products(s), which are captured by the capture reagents around the secreting cell.

Following the culture period, the cells are washed away, leaving their secretory product retained on the membrane. In some embodiments, the cells can remain on the detection surface.

A detection reagent bound to a detection particle is added to bind to the plate-bound secretory product (the detection and capture antibodies specific for secretory products. A detection particle is either directly labeled with a fluorochrome (e.g., FITC, PE or texas red) or has a characteristic spectral feature. (e.g., different spectral features for different cellular products). Directly labeled detection antibodies associated with detection particles are particularly useful. Alternatively, an antibody is added that is specific for the analyte and a secondary antibody is attached to the detection particle. Coupling of detection antibody to the detection particle by biotin-streptavidin can also be used.

There are a variety of means for visualizing the detection particles using a detection reagent such as: a) immune fluorescence if the secondary (or tertiary) reagent was a fluorochrome or b) a characteristic spectral feature (e.g., different “colors” for different analytes) visualized by light microscopy. Each “spot” of colored substrate corresponds to one cell producing or secreting the product. The difference in number of spots between antigen stimulated wells and the number of spots in the negative control wells (with medium alone or irrelevant antigen) establishes the “signal” to be analyzed (usually there is no spot formation in the negative control). Sizes of the spots correspond to the quantity of product secreted, the number of spots establishes the frequency of antigen specific cells in the cell population tested.

In other embodiments, methods can be used to use particles directly labeled with detection secondary antibodies. It is analogous to the indirect method described here, with the difference that interaction of the streptavidin-labeled particles with biotinylated secondary antibodies takes place prior to their interaction with the cytokine-primary coating antibody complex on the surface. The method is based on the interaction of streptavidin with its natural ligand, biotin, is practically irreversible; the equilibrium binding constant for this interaction exceeds the magnitude of 10¹¹ M⁻¹.

Particles of each individual type (color) covalently conjugated with streptavidin or its analog, avidin, are premixed with the biotin-labeled secondary detection antibody (reagent) specific to each secreted product. After the reaction is completed, the excess of unbound biotinylated antibodies is removed by the addition of agarose beads coated with streptavidin.

Agarose beads with the bound excessive biotinylated antibodies are then removed by centrifugation. Particles of different color, each labeled with single type of the secondary detection antibody (reagent) through streptavidin-biotin interaction, are incubated with the surface-bound secreted product similar to the way directly conjugated particles are used. Several particle types can be used either simultaneously or in a number of consecutive steps.

### Specific Embodiments

B-Cell ELISPOT Materials
1) MultiScreen_{HTS™} Filter Plates for ELISPOT (Millipore, Billerica, MA Cat. No. MSIPS4W).
2) Recombinant human IL-2 for polyclonal stimulation: Synonyms: T-cell growth factor (TCGF), Aldesleukin IL-2 is a powerful immunoregulatory lymphokine produced by T-cells in response to antigenic or mitogenic stimulation. IL-2/IL-2R signaling is essential for the immune response. IL-2 stimulates growth and differentiation of B-cells, NK cells, lymphokine activated killer cells, monocytes, macrophages and oligodendrocytes. (Peprotech, Rocky Hill, NJ; Cat. No. 200-02).
3) R848 for polyclonal stimulation: R848 is an imidazoquinoline compound with potent anti-viral activity. This low molecular weight synthetic molecule activates immune cells via the TLR7/TLR8 MyD88-dependent signaling pathway. Recently, R848 was shown to trigger NF-κB activation in cells expressing murine TLR8 when combined with poly(dT) (Enzo Life Sciences, Farmingdale, NY; Cat. No. ALX-420-038)
4) β-mercaptoethanol for polyclonal stimulation: (Sigma-Aldrich, St. Louis, MO; Cat. No. M7154)
5) Detection antibodies for B-cell ELISPOT—Anti-Human IgG Fc PAN (1,2,3,4), biotin-conjugated (Hybridoma Reagent Laboratory, Baltimore, MD; Cat. No. HP6043B)
6) Detection antibodies for B-cell ELISPOT—Anti-Human IgM, biotin-conjugated (Hybridoma Reagent Laboratory, Baltimore, MDl Cat. No. HP6083B).
7) Streptavidin-AP (Alkaline Phosphatase) (Sigma-Aldrich, St. Louis, MO; Cat. No. S2890).
8) Vector Blue Alkaline Phosphatase Substrate Kit; (Vector Laboratories, Burlingame, CA; Cat. No. SK-5300).
9) RPMI Media 1640 for cell culture with L-Glutamine: (Invitrogen, Grand Island, NY; Cat. No. 11875).
1-) Fetal bovine serum (FBS): (Invitrogen, Grand Island, NY; Cat. No. 16140).

Antibody ELISA
1) Immuno 96 MicroWell™ Solid Plates: (Thermo Scientific, Nunc, Rochester, NY; Cat. No. 456537).
2) Self antigens: (purified or from commercial sources, e.g., Novus Biologicals, Littleton, CO.).
3) Detection antibodies for B-cell ELISA—Anti-Human IgG Fc PAN (1,2,3,4), biotin-conjugated: (Hybridoma Reagent Laboratory, Baltimore, MD; Cat. No. HP6043B).
4) Fetal bovine serum (FBS): (Invitrogen, Grand Island, NY; Cat. No. 16140).
5) Streptavidin-HRP (Horseradish Peroxidase): (BD Pharmingen, San Jose, CA; Cat. No. 557630).
6) TMB (3,3′,5,5′-Tetramethylbenzidine) substrate: (eBioscience, San Diego, CA; Cat. No. 00-4201-56).

Detection of Anti-Self Antibodies in the Serum or Plasma

Despite detecting self-antigen-specific lymphocytes in the blood, antibodies that have already been secreted by these cells in vivo can be detected in the serum/plasma.

ELISA plates are coated with self-antigen. A sample of serum or plasma is collected and applied to the coated plates. After suitable incubation time allowing for sufficient binding of antibodies to the coated antigen, the presence of anti-self antibodies is detected by enzyme detection. Antibodies measured include IgM, IgD, IgG and IgG isotypes.

Detection of B-Lymphocyte Products

In certain embodiments of this invention, detection of B-lymphocyte products can be carried out using one of a variety of methods. Such methods include fluorometric spot assays (“Fluorospot™” Cellular Technology Ltd. Shaker Heights, OH), enzyme-linked spot (ELISPOT) assays, enzyme-linked immunosorbent assays (ELISA), immunoblotting (e.g., dot blot technique), polymerase chain reaction (PCR), and real-time PCR (RT-PCR), Radioimmunoassays (RIA), cytokine bead arrays, protein arrays, bead aggregation assays, intracellular cytokine staining, immunoprecipitation assays, immunohistochemistry, in situ cytokine detection on slides, cell screening or ultrasensitive densitometry by nanoparticle-modified aptamers.

ELISPOT Assays

According to embodiments of this invention, enzyme linked immune spot (“ELISPOT” assays can be carried out using methods described in U.S. Pat. Nos. 7,598,093 and 8,088,630, (incorporated herein fully by reference. Analyzers and methods from Cellular Technology Ltd (Immunospot® is a Registered Trademark of Cellular Technology Ltd. (“CTL”), Shaker Heights, OH). Analyzers and methods can be obtained from CTL.

Self-antigen-specific antibodies can be detected and measured in serum using ELISA, cytokine bead arrays, protein arrays and test strips. Antibodies detected can include IgA, IgD, IgE, IgG and IgM and their isotypes. Detection of B-lymphocytes in the blood includes measuring spontaneous production of antibodies by white blood cells after their isolation from the blood. B-lymphocyte ELISPOT assays, ELISA, cytokine bead arrays and protein array assays can be used. B-lymphocyte stimulation can be carried out by exposing B-cells to CNS antigens or by polyclonal stimulation by IL-2, R-848 and β-mercaptoethanol or other method commonly used to stimulate polyclonal production of B-lymphocytes.

### Examples of Specific Autoimmune Diseases

Myasthenia Gravis

Myasthenia gravis (MG) is a neuromuscular disorder that leads to weakness of voluntary skeletal muscles and can lead to myasthenic crisis and loss of respiratory regulation. One cause may be due to antibodies directed against acetylcholine receptors, which are the receptors responsible for normal neuromuscular transmission in voluntary or skeletal muscles. About 85% of people with MG have antibodies against the ACh receptor (AChR) in their blood. The antibodies (Y-shaped protein that immune cells, called B cells, use to attack bacteria and viruses) target and destroy many of the AChRs on cells of the muscle. Consequently, the muscle's response to repeated nerve signals declines with time, and the muscles become weak and can eventually disappear.

Human leukocyte antigens (HLAs) are associated with myasthenia gravis. These inclue HLA-B8, DRw3, and DQw2. MuSK antibody-positive myasthenia is associated with clusters of genes inherited together, including DR14 and DQ5.

Muscular Dystrophy

Muscular dystrophy is a group of diseases that cause progressive weakness and loss of muscle mass. In muscular dystrophy, abnormal genes (mutations) interfere with the production of proteins needed to form healthy muscle. When a muscle is damaged, its cells release an enzyme called creatin kinase. Mutations include those of the enzyme beta 1,3-N-acetylgalactosaminyltransferase 2 (B3GALNT2). Mutations in the B3GALNT2 enzyme impairs the glycosylation of alpha-dystroglycan, which causes congenital muscular dystrophy. Additionally, mutations of the sarcolemmal protein Merosin gene also is associated with muscular dystrophy.

There are many kinds of muscular dystrophy. Symptoms of the most common variety begin in childhood, mostly in boys. Other types don't surface until adulthood.

There are several forms of muscular dystrophy, including Duchenne's MD, Becher MD.

Duchenne's Muscular Dystrophy

In Duchenne's MD (DMD), symptoms include frequent falls, difficulty rising from a lying or sitting position, trouble running and jumping, waddling gait, walking on the toes, large calf muscles, muscle pain and stiffness, learning disabilities, or delayed growth. The genetic abnormalities in DMD and Becker muscular dystrophy are X-linked mutations in the dystrophin gene. Two-thirds of patients have large intergenic deletions or duplications. These lead to loss of the protein dystrophin, which connects the cytoskeleton of a muscle fiber to the surrounding extracellular matrix through the cell membrane. Loss of dystrophin leads to loss of stabilization of muscles and progressive weakness.

Becker Muscular Dystrophy

Signs and symptoms are similar to those of Duchenne muscular dystrophy, but tend to be milder and progress more slowly. Symptoms generally begin in the teens but might not occur until the mid-20s or later.

Other Types of Muscular Dystrophy

Some types of muscular dystrophy are defined by a specific feature or by where in the body symptoms begin. Examples include:
Myotonic. This is characterized by an inability to relax muscles following contractions. Facial and neck muscles are usually the first to be affected. People with this form typically have long, thin faces; drooping eyelids; and swanlike necks.
Facioscapulohumeral (FSHD). Muscle weakness typically begins in the face, hip and shoulders. The shoulder blades might stick out like wings when arms are raised. Onset usually occurs in the teenage years but can begin in childhood or as late as age 50.
Congenital. This type affects boys and girls and is apparent at birth or before age 2. Some forms progress slowly and cause only mild disability, while others progress rapidly and cause severe impairment.
Limb-girdle. Hip and shoulder muscles are usually affected first. People with this type of muscular dystrophy might have difficulty lifting the front part of the foot and so might trip frequently. Onset usually begins in childhood or the teenage years.

Systemic Lupus Erythematosus

Systemic lupus erythematosus (SLE) is a disorder characterized by antibodies directed to endothelial cells in blood vessels. Patients with SLE can experience blood clots and inflammation of blood vessels or vasculitis, inflammation of the heart, or pericarditis, heart attack, stroke, memory changes, behavioral changes, seizures, inflammation of lung tissue and the lining of the lung, or pleuritis, kidney inflammation, decreased kidney function, and kidney failure. In extreme cases, SLE can cause diffuse interstitial coagulation (DIC), where interstitial fluid actually clots, shutting down all functions of the affected organs. This is often a fatal condition.

Diagnosis of SLE is generally made based on a patient's history and symptomology. Assays for antinuclear antibodies (ANA) is a sensitive test, but healthy subjects may have elevated ANA. Antiphospholipid antibodies, anti-Smith, anti-endothelial cell antibodies (EACAs), and anti-nucleosome antibodies, and anti-double-strand DNA antibodies are also associated with SLE. Additionally, serum high-sensitivity cardiac troponin I (hs-cTnI) helps identify SLE patients with subclinical atherosclerosis.

According to a recent publication, Li et al., Sci Rep. 2022 Feb. 1; 12(1):1687 doi: 10.1038/s41598-022-05807-6, the sensitivity, specificity, sensitive likelihood ratio and specific likelihood ratio of indicators in SLE were determined by receiver-operator characteristic (ROC) curve after measurement of ANA and ANAs by indirect immunofluorescence (IIF) and immunoblotting, respectively. ROC analysis showed that the specificity of ANA titer ≥1+, ≥2+ and ≥3+ for SLE was estimated to be 81.29%, 90.69% and 96.52% respectively, with a increased titer-specific likelihood ratio (5.16, 9.29 and 19.60, respectively). The specificity of the number of positive-AAbs ≥1, ≥2 and ≥3 in ANAs for SLE was estimated to be 80.42%, 94.95% and 99.3% respectively, with a increased number-specific likelihood ratio (4.8, 15.26 and 72.48, respectively). The estimated sensitivity of the number of positive-AAbs ≥3, AnuA and anti-rRNP was higher than that of anti-Sm (p<0.01) (50.68%, 41.89% and 31.76% vs. 16.89%, respectively), while there was no significant difference in their specificity (99.3%, 99.74% and 99.56% vs. 99.74%, respectively) (p>0.05). High titers of ANA and the presence of multiple AAbs in ANAs are highly specific for SLE and highly suggestive of SLE. The likelihood of SLE can be assessed by ANA titer and the number of positive-AAbs in ANAs.

Treatments for SLE include anti-inflammatory drugs help treat pain or fever, antimalarials, which are used to prevent and treat malaria, have been found to be useful for treating fatigue, joint pain, skin rashes, and inflammation of the lungs caused by lupus. These drugs may also prevent flares from recurring, corticosteroids help to lower inflammation in the body, Immunosuppressants help suppress or curb the overactive immune system, and they may be given by mouth or by IV infusion. The risk for side effects increases with the length of treatment. Additionally, B-lymphocyte stimulator (BlyS) protein inhibitor, a type of biologic medication, can help reduce the activation and life span of abnormal B-cells in the body, which may help control lupus.

Grave's Disease

Grave's disease (GD), also known as toxic diffuse goiter is an immune system disorder resulting in overproduction of thyroid hormone (hyperthyroidism). Symptoms may include weight loss, despite an increased appetite, rapid or irregular heartbeat, nervousness, irritability, trouble sleeping, fatigue, shaky hands, muscle weakness, sweating or trouble tolerating heat, frequent bowel movements, and an enlarged thyroid gland (called a goiter). Additionally, some patients experience eye problems (Grave's opthalopathy (GO), including bulging eyes, gritty, irritated eyes, puffy eyes, light sensitivity, pressure or pain in the eyes or blurred or double vision.

These symptoms can start before or at the same time as symptoms of hyperthyroidism. Rarely, GO can develop after Graves' disease has been treated. You can develop GO even if your thyroid function is normal. Most people have mild symptoms.

The cause of Grave's disease may be due to an antibody called thyroid-stimulating immunoglobulin (TSI), which acts like thyroid-stimulating hormone (TSH). TSI causes the thyroid to overproduce thyroid hormone. Detection of TSI or thyroid stimulating hormone receptor (TSHR) can be used as a diagnostic tool. The recombinant human TSHR consists of 404 amino acids and predicts a molecular mass of 46.1 kDa. It migrates as an approximately 56.6 KDa band in SDS-PAGE under reducing conditions. Formulation Lyophilized from sterile 50 mM Tris, 150 mM NaCl, 10% Glycerol, 1 mM TCEP, pH 7.5.

For detecting Grave's disease, recombinant TSHR can be obtained from Novus biologicals having the amino acid sequence:

SEQ ID NO: 1
MRPADLLQLVLLLDLPRDLGGMGCSSPPCECHQEEDFRVTC
KDIQRIPSLPPSTQTLKLIETHLRTIPSHAFSNLPNISRI
YVSIDVTLQQLESHSFYNLSKVTHIEIRNTRNLTYIDPDA
LKELPLLKFLGIFNTGLKMFPDLTKVYSTDIFFILEITDN
PYMTSIPVNAFQGLCNETLTLKLYNNGFTSVQGYAFNGTK
LDAVYLNKNKYLTVIDKDAFGGVYSGPSLLDVSQTSVTAL
PSKGLEHLKELIARNTWTLKKLPLSLSFLHLTRADLSYPS
HCCAFKNQKKIRGILESLMCNESSMQSLRQRKSVNALNSP
LHQEYEENLGDSIVGYKEKSKFQDTHNNAHYYVFFEEQED
EIIGFGQELKNPQEETLQAFDSHYDYTICGDSEDMVCTPK
SDEFNPCEDIMGYKFLRIVVWFVSLLALLGNVFVLLILLT
SHYKLNVPRFLMCNLAFADFCMGMYLLLIASVDLYTHSEY
YNHAIDWQTGPGCNTAGFFTVFASELSVYTLTVITLERWY
AITFAMRLDRKIRLRHACAIMVGGWVCCFLLALLPLVGIS
SYAKVSICLPMDTETPLALAYIVFVLTLNIVAFVIVCCCY
VKIYITVRNPQYNPGDKDTKIAKRMAVLIFTDFICMAPIS
FYALSAILNKPLITVSNSKILLVLFYPLNSCANPFLYAIF
TKAFQRDVFILLSKFGICKRQAQAYRGQRVPPKNSTDIQV
QKVTHEMRQGLHNMEDVYELIENSHLTPKKQGQISEEYMQ
TVL.

Attaching this protein or an immunologically similar portion to the surface of a glass, plastic or membrane in vitro is a useful indicator of the presence of anti TSHR antibodies. Subsequently, a sample of PBMCs from a subject suspected of having Grave's disease is introduced onto the surface. If B cells in the PBMCs are programmed to produce anti TSHR antibodies, secretion of those antibodies can be detected using ELISPOT or ELISA assays.

Treatment of Grave's disease can include medicines, radioiodine therapy, or thyroid surgery. Medicines include use of antagonists of Beta adrenergic receptors (beta-blockers), antithyroid therapy using methimazole or propylthiouracil. Radiation therapy can be accomplished by use of radioactive iodine-131.

Rheumatoid Arthritis

Genetic markers for rheumatoid arthritis include the proteins PD-1/PD-L1/TIGIT/CTLA-4, HLA-DR4, HLA-DRB1, tyrosine phosphatase 22 (PTPN22), rheumatoid factor (RF), anti-cyclic citrullinated peptide (anti-CCP), and STAT4.

Detection of RA can be accomplished by attaching one or more of the above genetic marker proteins or an antigenically similar portion of the protein onto a suitable assay plate, such as a plastic or glass surface, or a polyvinyl membrane. A sample of PBMCs is then introduced onto the surface, and the B cells in the PBMCs secrete antibodies directed to the genetic marker(s). Secreted antibodies then bind to the marker protein, and then a suitable anti-IgG antibody, coupled to an enzyme, colored product, or radioactive isotope is added. The RA antibody produced by the PBMCs coupled to the anti-IgG molecule is then visualized by use of a microscope. The appearance of spots indicates the locations where the B cells secrete the antibodies. The intensity and number of spots seen are indicators of the magnitude of the antibody response.

Once a diagnosis is made, treatments include use of anti-tumor necrosis factor (anti-TNF; enantercept, infliximab, or adalimumab), and symptoms are monitored.

### EXAMPLES

This disclosure is further illustrated by the following examples, which are not considered limiting to the scope of this invention. Rather, they are used to point out certain embodiments. Persons of skill in the art can use the descriptions and teachings herein to produce other embodiments that are within the scope of this invention. All such embodiments are considered to be part of this invention.

### Example 1: Detection of Self-Antigen B-Cells and Antibodies from Patients with Grave's Disease

Filter plates are coated with thyroid stimulating hormone receptor (TSHR) antigen or an immunological equivalent of SEQ ID NO. 1. PBMCs are isolated from the blood by density gradient centrifugation and transferred to the plates as described in U.S. Pat. No. 7,598,093. After a suitable incubation time, allowing sufficient secretion and binding of antibodies to the coated antigen, the production of anti-self-antibodies is detected by enzyme-linked immunoassay detection. One can pre-incubate PBMCs with a polyclonal activator prior to introducing the cells into the assay plate Antibodies measured included IgM and IgG.

### Example 2: Detection of Self-Antigen B Cells and Antibodies from Patients with Muscular Dystrophy

Peripheral blood mononuclear cells (PBMCs) were isolated from the blood by density gradient centrifugation. The B cells contained in the sample are either used directly (Direct B Cell Test) or are polyclonally stimulated with IL-2, R-848 and β-mercaptoethanol for 3 days (Indirect B Cell Test) and subsequently transferred to filter plates that have been coated with the self-antigens beta 1,3-N-acetylgalactosaminyltransferase 2 (B3GALNT2), or dystrophin. The cells are then incubated on the plate for 24 h. The production of self-antigen-specific antibodies and binding to the antigen on the plate are detected by colorimetric enzyme detection. Antibodies measured include IgG.

### Example 3: Detection of Self-Antigen B Cells and Antibodies from Patients with Myasthenia Gravis

Using methods described herein, Myasthenia gravis antibodies can be detected by attaching acetylcholine receptors (ACHr) to the surface of an assay plate. A sample of PBMCs, either used without polyclonal activator (Direct B Cell Test) or after incubation with a polyclonal activator (Indirect B Cell Test) is deposited into the surface of the plate. After a suitable time, autoimmune antibodies are secreted, bind to the antigen, and subsequently, detector molecules can be used to bind to the antigen-antibody complex and can be visually quantified.

### Example 4: Detection of Self-Antigen B Cells and Antibodies from Patients with Systemic Lupus Erythematosus (SLE)

Using methods described herein, SLE can be diagnosed by detecting antinuclear antibodies (ANA), antiphospholipid antibodies, anti-Smith, anti-endothelial cell antibodies (EACAs), anti-nucleosome antibodies, anti-double-strand DNA antibodies and cardiac troponin I (hs-cTnI). An antigen is attached to an assay surface, and PBMCs, either used directly (Direct B Cell Test) or after polyclonal activation (Indirect B Cell Test) are placed over the antigen. Antibodies secreted by B cells bind to the endothelial cell antigens and are detected using an anti-IgG antibody or other detection method.

### Example 5: Detection of Self-Antigen B Cells and Antibodies from Patients with Rheumatoid Arthritis

Using methods described herein, markers for rheumatoid arthritis are attached to an assay plate. These markers include the proteins PD-1/PD-L1/TIGIT/CTLA-4, HLA-DR4, HLA-DRB1, tyrosine phosphatase 22 (PTPN22), rheumatoid factor (RF), anti-cyclic citrullinated peptide (anti-CCP), and STAT4. A sample of PBMCs, either used directly (Direct B Cell Test) or after polyclonal activation (Indirect B Cell Test) are placed in the well. Antibodies secreted by B cells bind to the self antigens and are detected using an anti-IgG antibody or other detection method.

### Example 6: Preparation of Purified Detection Particles Labeled with Streptavidin and Biotinylated Detection Antibodies

Using methods described herein, a bead array method includes use of detection particles having detection reagents thereon. In an embodiment a nanoparticle labeled with streptavidin is mixed with biotinylated detection reagent comprising biotin coupled to a detection reagent. These are incubated together for sufficient time to permit the biotin of biotinylated detection reagent to bind streptavidin on the nanoparticle thereby forming biotinylated streptavidin detection particle complex. Some free biotinylated detection reagent is free in solution. Then, an agarose particle having streptavidin thereon is added to the mixture and incubated for a further period of time. The resulting solution contains complexes and agarose/biotinylated detection reagent complexes. Little or no free biotinylated detection reagent is present in the solution. Next, a centrifugation step results in sedimentation of agarose/biotinylated detection reagent complexes at, leaving biotinylated streptavidin detection particle complexes free in solution. Subsequently, complexes can be used in detection particle, bead array-based assays.

### Example 7: Connective Tissue Diseases

Using methods described herein, connective tissue autoimmune diseases can be detected using one or more of the antigens:
a) ANA screen by IFA with reflex to ENA for all positive samples,
b) ENA identification by immunoassay (dsDNA, chromatin, ribosomal-P protein, SS-A, SS-B, Sm, Sm/RNP, RNP, Scl-70, Jo-1, and centromere B)
c) RF, CCP by immunoassay (IgG, IgM and IgA)
d) For autoimmune myositis profile (Mi-2, Ku, PM-Scl100, PM-Sc175, Jo-1, SRP, PL-7, PL-12, EJ, OJ, Ro-52)
e) For systemic sclerosis profile (Scl-70, CENP A, CENP B, RP11, RP155, Fibrillarin, NOR90, Th/To, PM-Scl100, PM-Sc175, Ku, PDGFR, Ro-52)

### Example 8: Autoimmune Vasculitis and APS

Using methods described herein, autoimmune fasculitis and APS can be detected using one or more of the antigens:
a) ANCA screen by IFA with reflex to immunoassay for all positive samples (pANCA, cANCA, X-ANCA)
b) PR3, MPO and GBM antibody identification by immunoassay
c) anti-endothelial cell antibodies (AECA)
d) anti-cardiolipin antibodies (aCL IgG and aCL IgM) or phospholipid antibodies (lupus anticoagulant, anti-beta-2 glycoprotein 1).

### Example 9: Antithyroid Autoantibodies

Using methods described herein, detection of antithyroid autoantibodies can be detected using. thyroid antigens: including one or more of THSA TPOAb, TRAb and TgAb.

### Example 10: Gastrointestinal Immune-Related Adverse Events

Using methods described herein, gastrointestinal autoantibodies can be detected using one or more of the following:
a) for celiac disease screen one can use (tTG and gliadin antibody)
b) for IBD screening, one can use one of more of ANCA, ASCA, OMP, DGP, A4-Fla2, or CBirl.

### Example 11: Autoimmune Hepatitis

Using methods described herein, hepatic autoantibodies for hepatitic and cholangitic patterns of injury can be detected using one or more of the following:
liver autoantibodies against one or more of actin, AMA M2, M2-3E, LKM-1, LC-1, SLA/LP, Sp100, PML, gp210, SLA/LP.

### Example 12: Autoimmune Nephritis

Using methods described herein, detection of autoimmune nephritis can be detected using one or more of PLA2R types M and N.

### Example 13: Autoimmune Dermatitis

Using methods described herein, autoimmune dermatitis can be detected using one or more of envoplakin, desmoglein 1, desmoglein 3, BP230-CF, BP180-NC16A-4. Prickle cell desmosomes, epidermal basement membrane, bullous pemphigoid antigens, keratin (filaggrin, “RA keratin”), gliadin (GAF-3X), or collagen type VII NC1.

### Example 14: Autoimmune Neuropathies

Using methods described herein, autoimmune neuropathies can be dectected using one or more of:
a) ganglioside profile (GM1, GM2, GM3, GD1a, GD1b, GT1b, GQ1b).
b) for neuronal antigens profile: (Amphiphysin/CV2.1, Recoverin, Hu, Yo, Ri, SOX1, titin)
c) for multiple sclerosis screen: (oligoclonal IgG bands, IgG index, Albumin ratio; isoelectrofocusing)
d) for paraneoplastic syndrome screen: (Yo, Hu, Ri, CV2, Ma1/2, amphiphysin, PNMA2, Zic4, GAD65, Tr, recoverin, SOX1, PCA-2, AGNA, CARPVIII; ELISA, Western blot, IFA)
e) for neurodegenerative diseases: one or more of Tau, Beta-amyloid, ApoE4, BASE1, neurogranin, pNf-H, or alpha-synuclein; ELISA.
f) for demyelination: one or more of NMOSD and M, AQP-4, MOG, flotillin, MAG, or gangliosides.

### Example 15: Therapies for Autoimmune Diseases

Treating autoimmune diseases may involve multi-faceted approaches including diet, lifestyle, and symptomatic treatments. Because autoimmune diseases involve production of anti-self antibodies produced by B cells, it can be helpful to treat patients with B cell specific therapies, such as use of rituximab, ocrelizumab, ofatimumab, belimumab, atacicept, epratuzumab, or anti-CD20 antibodies. These deplete B cells and thereby decrease the production of anti-self antibodies.

I claim:

1. A method for detecting an autoimmune antibody in a patient suspected of having an autoimmune disease, comprising:
a) preparing an assay plate for in vitro detection;
b) obtaining a sample of blood from said patient;
c) preparing peripheral blood mononuclear cells (PBMCs) from said sample;
d) attaching a self-antigen to the surface of said assay plate in cell culture medium;
e) placing said sample of PBMCs onto the assay plate;
f) permitting B cells to secrete anti-self-antibodies; and
g) detecting the presence of anti-self-antibodies bound to said antigen.

2. The method of claim 1, further comprising: exposing said PBMCs to a polyclonal activator being R848 and/or IL-2, and/or β-mercaptoethanol.

3. The method of claim 1, further comprising:
h) pre-conditioning an assay membrane with an antibody specific for a genetically-encoded hexahistidine (6×His) affinity tag.

4. The method of claim 1, where said self-antigen is one or more antigens associated with a connective tissue disease:
a) ANA screen by IFA with reflex to ENA for all positive samples;
b) ENA identification by immunoassay of dsDNA, or chromatin, or ribosomal-P protein, or SS-A, or SS-B, or Sm, or Sm/RNP, or RNP, or Scl-70, or Jo-1, or centromere B;
c) RF, or CCP by immunoassay for IgG, or IgM or IgA;
d) For autoimmune myositis profile antigens being Mi-2, or Ku, or PM-Sc100, or PM-Sc175, or Jo-1, or SRP, or PL-7, or PL-12, or EJ, or OJ, or Ro-52; and
a) For systemic sclerosis profile (Scl-70, or CENP A, or CENP B, or RP11, or RP155, or fibrillarin, or NOR90, or Th/To, or PM-Sc100, or PM-Sc175, or Ku, or PDGFR, or Ro-52).

5. The method of claim 1, where said self antigen is one or more antigens associated with autoimmune fasculitis and APS:
a) ANCA screen by IFA with reflex to immunoassay for all positive samples for antigens pANCA, or cANCA, or X-ANCA;
b) PR3, or MPO and or GBM antibody identification by immunoassay;
c) anti-endothelial cell antibodies (AECA);
d) anti-cardiolipin antibodies (aCL IgG and aCL IgM) or
e) phospholipid antibodies for antigens lupus anticoagulant, or anti-beta-2 glycoprotein 1.

6. The method of claim 1, where said self antigen is one or more antigens associated with antithyroid autoantibody immune disease, for the antigens THSA or TPOAb, or TRAb or TgAb.

7. The method of claim 1, where Grave's disease is detected using THSA, having the sequence of SEQ. ID. No. 1, or an immunologically equivalent THSA.

8. The method of claim 1, where said self antigen is one or more antigens associated with gastrointestinal autoimmune disease:
a) for celiac disease screen one can use tTG or gliadin antibody;
b) for IBD screening, one of more of ANCA, or ASCA, or OMP, or DGP, or A4-Fla2, or CBirl.

9. The method of claim 1, where said self antigen is one or more antigens associated with hepatic autoantibodies for hepatitic and cholangitic patterns of injury being actin, or AMA M2, or M2-3E, or LKM-1, or LC-1, or SLA/LP, or Sp100, or PML, or gp210, or SLA/LP.

10. The method of claim 1, where said self antigen is one or more antigens associated with autoimmune nephritis, the antigens being PLA2R types M or PLA2R type N.

11. The method of claim 1, where said self antigen is one or more antigens associated with autoimmune dermatitis being envoplakin, or desmoglein 1, or desmoglein 3, or BP230-CF, or BP180-NC16A-4, or Prickle cell desmosomes, or epidermal basement membrane, or bullous emphigoid antigens, or keratin (filaggrin, “RA keratin”), or gliadin (GAF-3X), or collagen type VII NC1.

12. The method of claim 1, where said self antigen is one or more antigens associated with autoimmune neuropathies being:
a) for ganglioside profile antigens being GM1, or GM2, or GM3, or GD1a, or GD1b, or GT1b, or GQ1b;
b) for neuronal antigens profile antigens being Amphiphysin/CV2.1, or Recoverin, or Hu, or Yo, or Ri, or SOX1, or titin;
c) for multiple sclerosis screen antigens being oligoclonal IgG bands, or IgG index, or Albumin ratio; or isoelectrofocusing;
d) for paraneoplastic syndrome screen, antigens being: Yo, or Hu, or Ri, or CV2, or Ma1/2, or amphiphysin, or PNMA2, or Zic4, or GAD65, or Tr, or recoverin, or SOX1, or PCA-2, or AGNA, or CARPVIII detected using ELISA, or Western blot, or IFA;
e) for neurodegenerative diseases: one or more of Tau, or Beta-amyloid, or ApoE4, or BASE1, or neurogranin, or pNf-H, or alpha-synuclein detected using ELISA;
f) for demyelination: one or more of NMOSD or M, or AQP-4, or MOG, or flotillin, or MAG, or gangliosides.

13. The method of claim 1, where said self antigen is one or more antigens associated with Grave's disease.

14. The method of claim 1, where said self antigen is one or more antigens associated with muscular dystrophy.

15. The method of claim 1, where said self antigen is one or more antigens associated with myasthenia gravis.

16. The method of claim 1, where said self antigen is one or more antigens associated with systemic lupus erythematosus.

17. The method of claim 1, where said self antigen is one or more antigens associated with rheumatoid arthritis.

18. A method for treating a patient having an autoimmune disease, comprising the steps:
a) providing a cell culture well having a surface and a cell culture medium therein;
b) attaching an autoimmune disease-specific antigen to said surface;
c) introducing a sample of peripheral blood mononuclear cells (PBMCs) into said cell culture medium;
d) permitting said PBMCs to produce an antibody against said autoimmune disease-specific antigen;
e) detecting the presence of said antibody using an anti-antibody specific reagent; and
f) if said antibody is detected in step e), administering to said patient an immune modulating agent.

19. The method of claim 17, wherein said immune modulating agent is a B-cell depleting agent, anti-CD20 antibody.

20. The method of claim 19, wherein said immune modulating agent is anti-CD20 antibody, glatiramer acetate, interferon beta-1a, interferon beta-1b, mitoxantrone, natalizumab, or FTY720 fingolimod.
